(19) United States
(12) Patent Application Publication
(10) Pub. No.: US 2024/0342115 A1
(43) Pub. Date: Oct. 17, 2024

(54) MICROSPHERE FORMULATIONS COMPRISING KETAMINE AND METHODS FORMAKING AND USING THE SAME

(71) Applicant: Oakwood Laboratories, LLC, Oakwood Village, OH (US)

(72) Inventors: Rachel Galaska, Willoughby, OH (US); Kelsey Kaht, Kent, OH (US); Tracy Richey, Stow, OH (US)

(21) Appl. No.: 18/756,907

(22) Filed: Jun. 27, 2024

Related U.S. Application Data
(63) Continuation-in-part of application No. 17/404,128, filed on Aug. 17, 2021.
(60) Provisional application No. 63/067,068, filed on Aug. 18, 2020.
(60) Provisional application No. 63/149,911, filed on Feb. 16, 2021.

(51) Int. Cl. A61K 31/135; A61K 9/16

(52) U.S. Cl. CPC A61K 31/135; A61K 9/1647

(57) ABSTRACT

Extended-release, injectable microsphere formulations comprising ketamine are provided. Methods for making and using the microsphere formulations are also provided.

### CROSS-REFERENCE TO RELATED APPLICATIONS

This application is a continuation in part of U.S. Nonprovisional patent application Ser. No. 17/404,128, filed on Aug. 17, 2021, which claims priority from U.S. Provisional Patent Application No. 63/067,068, filed on Aug. 18, 2020, and U.S. Provisional Patent Application No. 63/149,911, filed on Feb. 17, 2021. This application also claims the benefit of U.S. Provisional Patent Application No. 63,510,512, filed on Jun. 27, 2023. Each of these applications is incorporated by reference herein in its entirety.

### BACKGROUND

Ketamine (chemical formula C₁₃H₁₆ClNO, IUPAC name 2-(2-chlorophenyl)-2-(methylamino)cyclohexan-1-one), characterized by the general structure:

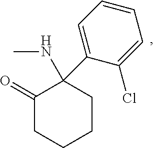 [SMILES: CNC1(c2ccccc2Cl)CCCCC1=O]

is an N-methyl-D-aspartate (“NMDA”) receptor antagonist. Ketamine has primarily been used for anesthesia in humans and animals, as well as for chronic pain and sedation. Ketamine is typically available commercially in liquid form for use as an immediate-acting injection.

Ketamine is a racemic mixture of two enantiomers, (S)-(+)-ketamine and (R)-(−)-ketamine. The (S)-(+) enantiomer, also known as esketamine, is significantly more potent as an NMDA receptor antagonist and anesthetic than is the (R)-(−) enantiomer, also known as arketamine.

Ketamine and its enantiomers have also been investigated for treatment of depression. The U.S. Food and Drug Administration (“FDA”) has approved esketamine for use, in conjunction with an oral antidepressant, for the treatment of treatment-resistant depression (“TRD”) and major depressive disorder (“MDD”) in adults. Specifically, the FDA approved Spravato® nasal spray. For the treatment of TRD, the manufacturer recommends that the drug be administered twice per week for the first four weeks, weekly for the next four weeks, then weekly or biweekly.

Ketamine is also used by recreational drug users and abusers. Ketamine is a Schedule III drug under the U.S. Drug Enforcement Agency's controlled substance classifications under the Controlled Substances Act. In part because of the significant possibility for diversion, Spravato® nasal spray is approved only for administration under the direct supervision of a healthcare provider. This requires a patient to make multiple trips per week to a doctor's office or hospital for the first four weeks, then weekly visits thereafter, making treatment inconvenient for the patient. Patients are also required to stay in the doctor's office or hospital for at least two hours after administration, adding to the patient's inconvenience. Until now, there has been a long felt yet unresolved need for a ketamine formulation that reduces the number of visits a patient needs to make to a provider's office to receive treatment, to reduce cost and inconvenience to the patient and the provider, while maintaining the ability to keep the drug in the hands of the healthcare providers to prevent diversion.

### SUMMARY

Microsphere formulations comprising ketamine are provided. The microsphere formulations comprise polymer microspheres, each polymer microsphere comprising: (i) ketamine; and (ii) a biodegradable polymer comprising a poly(lactide) (“PLA”) polymer. Each polymer microsphere may comprise a drug load of between about 10 wt/wt % to about 20 wt/wt %, and the polymer microspheres may have a particle size of from about 80 μum (D₅₀) to about 100 μm (D₅₀). In some aspects, the polymer microspheres are characterized by a plurality of internal emulsions, each emulsion comprising water and a surfactant. In some aspects, the polymer microspheres may be subjected to dehydration, in which case the polymer microspheres are characterized by a plurality of internal macrovoids.

In some aspects, the polymer microspheres are double emulsified. A method for making double emulsified polymer microspheres is provided, the method comprising: (i) contacting ketamine with a biodegradable PLA polymer in the presence of a solvent to form an organic component and providing the organic component to a first homogenizer; (ii) providing an inner aqueous component comprising water and a first surfactant to the first homogenizer; (iii) homogenizing the organic component with the inner aqueous component to form a primary emulsion; (iv) providing the primary emulsion to a second homogenizer at a first flow rate; (v) providing a continuous phase comprising water and a second surfactant to the second homogenizer at a second flow rate; (vi) homogenizing the primary emulsion and the continuous phase; and (iv) removing the solvent to form the polymer microspheres, wherein each of the formed polymer microspheres incorporates at least a portion of the inner aqueous component in the form of a plurality of emulsions. In some aspects, the polymer microspheres may be subjected to dehydration, in which case the polymer microspheres are characterized by a plurality of internal macrovoids.

In another aspect, a method for treating depression, including TRD and/or MDD, is provided. The method may comprise administering to a patient in need thereof a microsphere formulation, the microsphere formulation comprising: polymer microspheres, each polymer microsphere comprising: (i) ketamine; and (ii) a PLA polymer, wherein each polymer microsphere comprises a drug load of between about 10 wt/wt % to about 20 wt/wt %, and wherein the polymer microspheres have a particle size of from about 80 μm (D₅₀) to about 100 μm (D₅₀). In some aspects, the microsphere formulation is administered to the patient by intramuscular or subcutaneous injection with a dosing schedule of about every thirty days.

In another aspect, a method for treating pain is provided. The method may comprise administering by intramuscular or subcutaneous injection to a patient in need thereof a microsphere formulation made according to the methods described herein.

In another aspect, use is disclosed of a microsphere formulation comprising polymer microspheres, each polymer microsphere comprising: (i) ketamine; and (ii) a PLA polymer, wherein each polymer microsphere comprises a drug load of between about 10 wt/wt % to about 20 wt/wt %, and wherein the polymer microspheres have a particle size of from about 80 μm (D₅₀) to about 100 μm (D₅₀), in the manufacture of a medicament for the treatment of depression.

In another aspect, a microsphere formulation comprising polymer microspheres, each polymer microsphere comprising: (i) ketamine; and (ii) a PLA polymer, wherein each polymer microsphere comprises a drug load of between about 10 wt/wt % to about 20 wt/wt %, and wherein the polymer microspheres have a particle size of from about 80 μm (D₅₀) to about 100 μm (D₅₀), is provided for use as a medicament for the treatment of depression.

### BRIEF DESCRIPTION OF THE FIGURES

FIG. 1 is a flow chart illustrating an example method for making a double-emulsified microsphere formulation.

FIGS. 2A and 2B are two photographs showing a comparison between polymer microspheres prepared using a double emulsion technique (FIG. 2A) and a single emulsion technique (FIG. 2B), each prior to dehydration.

FIG. 3 is a graph showing an amount of ketamine released in vitro over time from three example double emulsified microsphere formulations and one example single emulsified formulation.

FIG. 4 is a graph showing an amount of ketamine released in vitro over time from an example double emulsified microsphere formulation.

FIG. 5 is a graph showing example results of a pharmacokinetics study in rats using microsphere formulations as described herein.

FIG. 6 is a graph showing an amount of ketamine released in vitro over time from several microsphere formulations prepared using a double emulsification technique.

FIG. 7 is a graph showing an amount of ketamine released in vitro over time versus a linear 30 day release from a microsphere formulation prepared using a double emulsification technique.

FIG. 8 is a graph showing example results of a pharmacokinetics study in dogs using large scale batches of microsphere formulations as described herein.

FIG. 9 is an inset graph depicting an expanded view of the first 0.5 day of the measurements shown in FIG. 8 and showing the burst release levels of ketamine.

FIG. 10 is a graph showing the in vitro-in vivo correlation of one of the pharmacokinetics study formulations referred to in FIG. 8.

FIGS. 11A and 11B are graphs showing a comparison of deconvoluted in vivo release versus in vitro release of the pharmacokinetics study formulations referred to in FIG. 8.

FIG. 12 is a graph showing simulated dosing of the pharmacokinetics study formulations referred to in FIG. 8 over three months on a linear scale.

FIG. 13 is a graph showing a comparison of the in vivo release of a claimed microsphere formulation in dogs versus the blood levels in humans resulting from nasal inhalation of the commercial product Spravato® nasal spray.

### DETAILED DESCRIPTION

Microsphere formulations comprising ketamine are provided. The microsphere formulations comprise polymer microspheres, each polymer microsphere comprising: (i) ketamine; and (ii) a PLA polymer, wherein each polymer microsphere comprises a drug load of between about 10 wt/wt % to about 20 wt/wt %, and wherein the polymer microspheres have a particle size of from about 80 μm (D₅₀) to about 100 μm (D₅₀). In some aspects, the polymer microspheres are characterized by a plurality of internal emulsions, each emulsion comprising water and a surfactant. In some aspects, the polymer microspheres may be subjected to dehydration, in which case the polymer microspheres are characterized by a plurality of internal macrovoids.

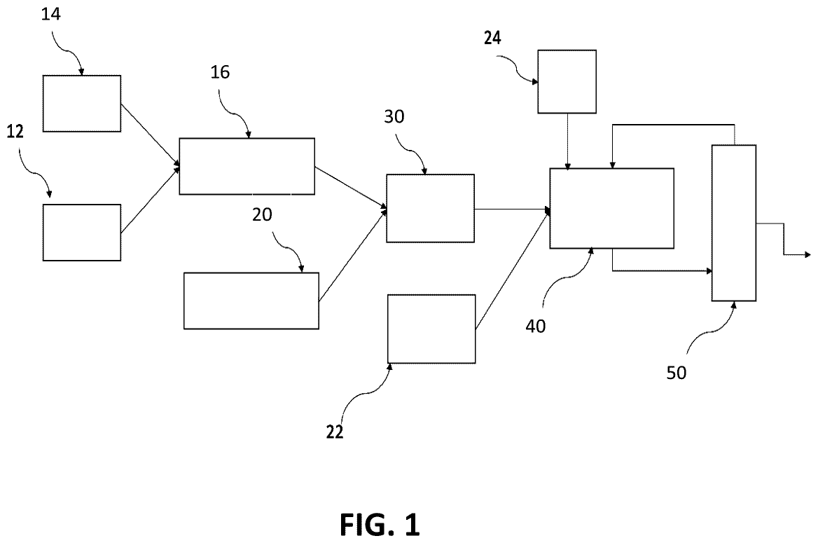

In some aspects, the polymer microspheres are double emulsified. With reference to FIG. 1, a method for making double-emulsified polymer microspheres is provided, the method comprising: (i) contacting ketamine with a biodegradable PLA polymer in the presence of a solvent to form an organic component and providing the organic component to a first homogenizer; (ii) providing an inner aqueous component comprising water and a first surfactant to the first homogenizer; (iii) homogenizing the organic component with the inner aqueous component to form a primary emulsion; (iv) providing the primary emulsion to a second homogenizer at a first flow rate; (v) providing a continuous phase comprising water and a second surfactant to the second homogenizer at a second flow rate; (vi) homogenizing the primary emulsion and the continuous phase; and (iv) removing the solvent to form the polymer microspheres, wherein each of the formed polymer microspheres incorporates at least a portion of the inner aqueous component in the form of a plurality of emulsions. In some aspects, the polymer microspheres may be subjected to dehydration, in which case the polymer microspheres are characterized by a plurality of internal macrovoids.

### API-Ketamine

In some aspects, the ketamine comprises a racemic mixture. In some aspects, the ketamine comprises esketamine to the exclusion of arketamine. Alternatively, the ketamine may comprise arketamine to the exclusion of esketamine.

In some aspects, the ketamine may comprise a pharmaceutically acceptable salt form or a free base form of any of ketamine, esketamine to the exclusion of arketamine, and arketamine to the exclusion of esketamine. Suitable salts may include hydrochloride, sulfate, acetate, phosphate, diphosphate, chloride, maleate, citrate, mesylate, nitrate, tartrate, gluconate, and the like. In other aspects, a complex salt may be used to decrease solubility, such as ketamine palmitate, ketamine benzoic acid, ketamine tosylic acid, ketamine camphor-sulfonic acid, and the like.

Unless otherwise noted, as used herein, the term “ketamine” is intended to include the racemic mixture as well as both of its individual enantiomers. In some aspects, the ketamine may be used in its racemic form. Alternatively, the ketamine may be used in its enantiomeric forms, such as in its “S” or “R” forms. An aspect may also include purified forms of the enantiomeric forms. For example, and without limitation, the “S” enantiomer to “R” enantiomer ratio may be from 51:49 up to 100:0 and every range included therein. Alternative aspects may comprise more purified forms of the “R” enantiomer over the “S” enantiomer. For example, and without limitation, the “R” enantiomer to “S” enantiomer ratio may be from 51:49 up to 100:0, and every range included therein. Each enantiomer may also exist in its (+) or (−) forms, such as in S(+) or S(−) forms. An alternative aspect is the use of a purified form of esketamine in which the ratio of S(+) to S(−) may be from 51:49 up to 100:0, and every range included therein. An alternate aspect is the use of a purified form of esketamine in which the ratio of S(−) to S(+) may be from 51:49 up to 100:0, and every range included there.

In one aspect, the ketamine consists of or consists essentially of(S)-ketamine base (esketamine base). In this context, “consists essentially of(S)-ketamine base (esketamine base)” means that the microsphere formulation does not contain a sufficient amount (which includes having a zero amount) of a second active pharmaceutical ingredient to have a measurable physiological effect on a condition known to be treatable by administration of such second active pharmaceutical ingredient. In one aspect, the microsphere formulation is exclusive of hydromorphone.

### Biodegradable Polymers-PLAs

In one aspect, the PLA may have an inherent viscosity (“IV”) between about 0.30 to about 1.8 dL/g, including from about 0.60 to about 0.72 dL/g, and including about 0.66 dL/g, about 0.67 dL/g, or about 0.72 dL/g. In another aspect, the PLA may have an IV of about 0.67 dL/g. In one aspect, the biodegradable polymer is an Ashland DL 07E PLA polymer have an IV of about 0.67 dL/g or about 0.72 dL/g.

As the phrase is used herein, a “poly(lactide) polymer” is to be distinguished from and does not include a poly(lactic-co-glycolic acid) polymer. In some aspects, copolymers are specifically excluded. In one aspect, PLGA polymers are specifically excluded.

In some aspects, the biodegradable polymers are ester end-capped. In some aspects, acid end-capped biodegradable polymers are specifically excluded.

### Organic Component-Solvents

The ketamine and the PLA may be dissolved in a solvent mixture to form an organic component. Suitable solvents may include methylene chloride (also known as dichloromethane or DCM), ethanol, ethyl acetate, acetic acid, acetone, acetonitrile, acetyl acetone, acrolein, acrylonitrile, allyl alcohol, 1,3-butanediol, 1,4-butanediol, 1-butanol, 2-butanol, tert-butanol, 2-butoxyethanol, n-butyl amine, butyl dioxitol acetate, butyraldehyde, butyric acid, 2-chloroethanol, diacetone alcohol, diacetyl, diethylamine, diethylene glycol diethyl ether, diethylene glycol dimethyl ether, diethylene glycol monobutyl ether, diethylene glycol monobutyl ether acetate, diethylene glycol monoethyl ether, diethylene glycol monoethyl ether acetate, diethylene glycol monomethyl ether, N,N-diethylnicotinamide, dimethyl sulfoxide, N,N-dimethylacetamide, N,N-dimethylformamide, 1,4-dioxane, 2-ethoxyethanol, 2-ethoxyethyl acetate, ethyl acetate, ethyl formate, ethylene glycol methyl ether acetate, formic acid, furfural, glycofurol, hexylene glycol, isobutanol, isopropyl alcohol, 2,6-lutidine, methyl acetate, methyl ethyl ketone, methyl isopropyl ketone, methyl propionate, N-methylpyrrolidone, morpholine, tert-pentanol, 2-picoline, 3-picoline, 4-picoline, piperidine, 1-propanol, propionaldehyde, propylene oxide, pyridine, pyrimidine, pyrrolidine, tetrahydrofuran, tetramethylurea, triacetin, triethylene glycol, trimethyl phosphate, and combinations thereof. In some aspects, the solvent comprises DCM, ethanol, ethyl acetate, or a combination of two or all of them. In some aspects, the solvent an about 5:1 (by volume) ratio of DCM:ethanol. In some aspects, the solvent an about 8.5:1 (by weight) ratio of DCM:ethanol.

### Inner Aqueous Component

In one aspect, the organic component is homogenized with an inner aqueous component to form a primary emulsion. In one aspect, the inner aqueous component comprises water. In one aspect, the inner aqueous component comprises water and a surfactant. In one aspect, the surfactant comprises polyvinyl alcohol (“PVA”). In some aspects, the inner aqueous component comprises PVA in an amount of about 0.35% to about 1.0% by weight in water. In some aspects, the inner aqueous component comprises PVA in an amount of about 0.35% by weight in water. In some aspects, the inner aqueous component comprises PVA in an amount of about 1.0% by weight in water.

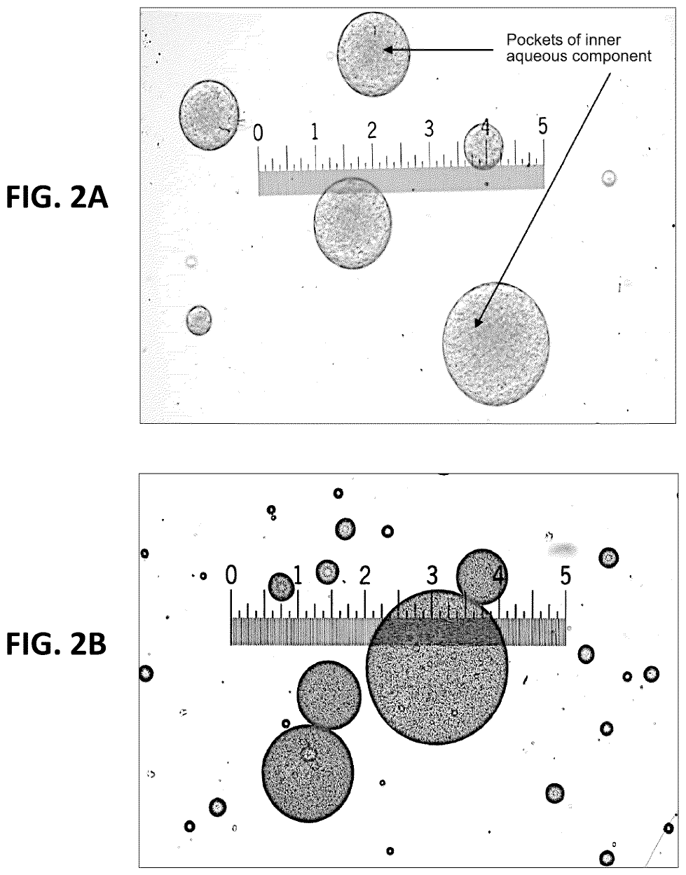

FIGS. 2A and 2B are two photographs showing a comparison between polymer microspheres prepared using a double emulsion technique (FIG. 2A) and a single emulsion technique (FIG. 2B), each prior to dehydration. A single emulsion microsphere technique is described in US20220054420A1. The double emulsified polymer microspheres are characterized in that each of the polymer microspheres incorporates a plurality of emulsions comprising water and the surfactant. In some aspects, the polymer microspheres may be subjected to dehydration, in which case the polymer microspheres are characterized by a plurality of internal macrovoids.

In one aspect, dehydration may be achieved by freeze drying, including by lyophilization or cryodesiccation, i.e., a low temperature dehydration process that involves freezing the polymer microspheres, lowering pressure, and removing the ice by sublimation. This is in contrast to dehydration methods that evaporate water using heat.

### Continuous Phase

The primary emulsion may be homogenized with a continuous phase comprising water and, optionally, a surfactant, such as PVA, to form a secondary emulsion. The surfactant component may be present in the continuous phase in an amount of about 0.35% to about 1.0% by weight in water. In one aspect, the surfactant component comprises PVA in an amount of about 0.35% by weight in water. In one aspect, the surfactant component comprises PVA in an amount of about 0.5% by weight in water. In one aspect, the surfactant component comprises PVA in an amount of about 1.0% by weight in water. The secondary emulsion may be subjected to solvent removal and washing processes to form the double emulsified polymer microspheres.

In some aspects, the primary emulsion flow rate to the second homogenizer may from about 10 mL/min to about 30 ml/min, including about 20 mL/min and about 25 mL/min. In some aspects, the continuous phase flow rate to the homogenizer may be about 2 L/min. Thus, in one aspect, the continuous phase:primary emulsion ratio may be from about 66:1 to about 200:1, including about 100:1 and about 80:1.

The continuous phase may be provided at room temperature or above or below room temperature. In some aspects, the continuous phase may be provided at about 40° C., about 37° C., about 35° C., about 30° C., about 25° C., about 20° C., about 15° C., about 10° C., about 5° C., about 0° C., and any range or value between any of those values.

### Homogenizer

In some aspects, the homogenization of the organic component and the inner aqueous component may be conducted in a high-speed homogenizer, e.g., in a T25 Ultra-turrax high-speed homogenizer operating, e.g., at 21,500 rpm for 30 seconds (e.g., run in 15 second intervals for two intervals) to form the primary emulsion. In other aspects, the homogenization of the organic component and the inner aqueous component may be conducted in a sonicator, e.g., a Q700 Sonicator (manufactured by Qsonica), or in a magic LAB® DISPAX-REACTOR® DR (manufactured by IKA).

In some aspects, the homogenization of the primary emulsion and the continuous phase may be conducted in an emulsifier or a homogenizer. For brevity, and because the methods are equally applicable to either, the phrase “homogenizer” contemplates a system or apparatus that can homogenize the primary emulsion and the continuous phase, emulsify the primary emulsion and the continuous phase, or both, which systems and apparatuses are known in the art. For example, in one aspect, the homogenizer is an in-line Silverson Homogenizer (commercially available from Silverson Machines, Waterside UK) or a Levitronix® BPS-i100 integrated pump system used, e.g., as described in U.S. Pat. No. 11,167,256, which is incorporated by reference herein in its entirety. In one aspect, the homogenizer is a membrane emulsifier. In one aspect, the homogenizer runs at an impeller speed of about 1,000 to about 4,000 revolutions per minute (“RPM”), including about 1,600 RPM.

### Particle Size

The polymer microspheres may be any size that is safely and efficaciously injectable by intramuscular or subcutaneous injection. In one aspect, the polymer microspheres may have a particle size greater than 60 μm (D₅₀) to about 110 μm (D₅₀), including between about 80 μm (D₅₀) and about 110 μm (D₅₀), and including about 90 μm (D₅₀). In one aspect, particle sizes of 60 μm or less are excluded. In one aspect, particle sizes of less than 80 μm (D₅₀) are excluded.

### Drug Load

The drug load of each polymer microsphere in a drug to polymer ratio, expressed as a percentage, may range from between about 10 wt/wt % to about 50 wt/wt %, from between about 10 wt/wt % to about 30 wt/wt %, from between about 10 wt/wt % to about 20 wt/wt %, or from between about 14 wt/wt % to about 15 wt/wt %.

### Extended Release

The microsphere formulations are characterized in that they have an in vivo duration of ketamine release of about 25 days to about 35 days, including about 30 days.

### Therapeutic Benefits

Possible conditions that may be treated using the microsphere formulations include depression, TRD, MDD, conditions involving excitotoxicity including neurodegenerative diseases and benzodiazepine withdrawal, pain, and other diseases or conditions that may be treated by the inhibition of action of the NMDA receptor.

In one aspect, depression, TRD, or MDD may be treated using the microsphere formulations, wherein the microsphere formulations are administered about every 25 days to about 35 days, including about every 30 days.

In another aspect, a method for treating depression, including TRD and/or MDD, is provided. The method may comprise administering to a patient in need thereof a microsphere formulation, the microsphere formulation comprising: polymer microspheres, each polymer microsphere comprising: (i) ketamine; and (ii) a PLA polymer, wherein each polymer microsphere comprises a drug load of between about 10 wt/wt % to about 20 wt/wt %, and wherein the polymer microspheres have a particle size of from about 80 μm (D₅₀) to about 100 μm (D₅₀). In some aspects, the microsphere formulation is administered to the patient by intramuscular or subcutaneous injection with a dosing schedule of about every 25 days to about 35 days, including about every 30 days.

In another aspect, a method for treating pain is provided. The method may comprise administering by intramuscular or subcutaneous injection to a patient in need thereof a microsphere formulation made according to the methods described herein.

In another aspect, use is disclosed of a microsphere formulation comprising polymer microspheres, each polymer microsphere comprising: (i) ketamine; and (ii) a PLA polymer, wherein each polymer microsphere comprises a drug load of between about 10 wt/wt % to about 20 wt/wt %, and wherein the polymer microspheres have a particle size of from about 80 μm (D₅₀) to about 100 μm (D₅₀), in the manufacture of a medicament for the treatment of depression.

In another aspect, a microsphere formulation comprising polymer microspheres, each polymer microsphere comprising: (i) ketamine; and (ii) a PLA polymer, wherein each polymer microsphere comprises a drug load of between about 10 wt/wt % to about 20 wt/wt %, and wherein the polymer microspheres have a particle size of from about 80 μm (D₅₀) to about 100 μm (D₅₀), is provided for use as a medicament for the treatment of depression.

The microsphere formulations are extended-release, injectable formulations for administration via intramuscular or subcutaneous injection and not intrathecally. In some aspects, the intramuscularly or subcutaneously injectable formulation may further include sodium carboxymethylcellulose, tween 80, and mannitol.

### EXAMPLES

### Example 1—General Preparation of Microsphere Formulations Comprising Ketamine Via a Double Emulsion Method

Microsphere Formation Phase. With reference to FIG. 1, an organic component 12 is formed by dissolving a biodegradable polymer (such as a PLA polymer) in an organic solvent (such as DCM, ethanol, or a combination thereof), followed by the addition of ketamine with mixing until completely dissolved. The organic component 12 is homogenized with an inner aqueous component (“IA component”) 14 comprising water and, optionally, PVA, in a high-speed homogenizer probe (such as a T25 Ultra-turrax, a Q700 Sonicator, or a magic Lab® DISPAX-REACTOR®) 16 to form a primary emulsion (“PE”). The PE is pumped into a homogenizer 30, such as an in-line Silverson Homogenizer or a Levitronix i100 (as described in U.S. Pat. No. 11,167,256), at a defined flow rate. The CP 20 comprising water and, optionally, PVA, is also pumped into the homogenizer 30 at a defined flow rate.

Microsphere Processing Phase. The formed or forming microspheres exit the homogenizer 30 and enter an SRV 40. Water 22 is added to the SRV 40 during microsphere formation to minimize the solvent level. The resulting suspension is mixed in the SRV 40 during the microsphere formation period. After the PE is exhausted, the CP and water flow additions are stopped, and the washing steps are initiated.

Solvent removal is achieved by washing the microspheres with ambient water 24 (i.e. 25° C.) and/or hot water (35-39° C.) and filtering them through a filter, e.g., a hollow fiber filter 50. Excess solvent is removed and discarded, and the filtered microspheres are returned to the SRV until the desired level of solvent is removed from the microsphere formulation.

The washed microspheres are collected on a filter membrane and freeze-dried overnight in a lyophilizer (Virtis) to remove moisture. The resulting microspheres are a free-flowing off-white bulk powder.

### Example 2—Preparation and Evaluation of a Low Inherent Viscosity (0.66 dL/g) PLA-Based Double Emulsion Microsphere Formulation

Batch No. 8: The organic component was formed by dissolving 7.0 g of an ester end-capped PLA Ashland Viatel 07 E polymer (IV=0.66 dL/g) in 39 g of DCM and 4.6 g of ethanol (5:1 ratio by volume), followed by the addition of esketamine (3.0 g) with mixing until completely dissolved. The organic component was homogenized with an IA component consisting of 1 mL of de-ionized water in a T25 Ultra-turrax high-speed homogenizer operating at 21,500 rpm for 30 seconds to form the PE.

The PE was pumped into a Levitronix i100 operating at 1,600 rpm at a rate of 30 mL/minute, along with a CP comprising water and 0.35% PVA, which was pumped at a rate of 2 L/min, for a CP:PE ratio of 66:1.

The formed or forming microspheres exited the homogenizer and entered an SRV. Deionized water was added to the SRV at 2 L/min. Solvent removal was achieved by washing the microspheres with ambient water (i.e., 25° C.) and hot water (35-39° C.) and filtering them through a hollow fiber filter.

The bulk suspension was collected via filtration and lyophilized to obtain a free-flowing powder with a yield of about 59%. The drug load was 16.5 wt/wt % (55% drug encapsulation efficiency based on a target drug load of 30 wt/wt %). The average particle size was 47 μm (D₁₀), 82 μm (D₅₀), 132 μm (D₉₀).

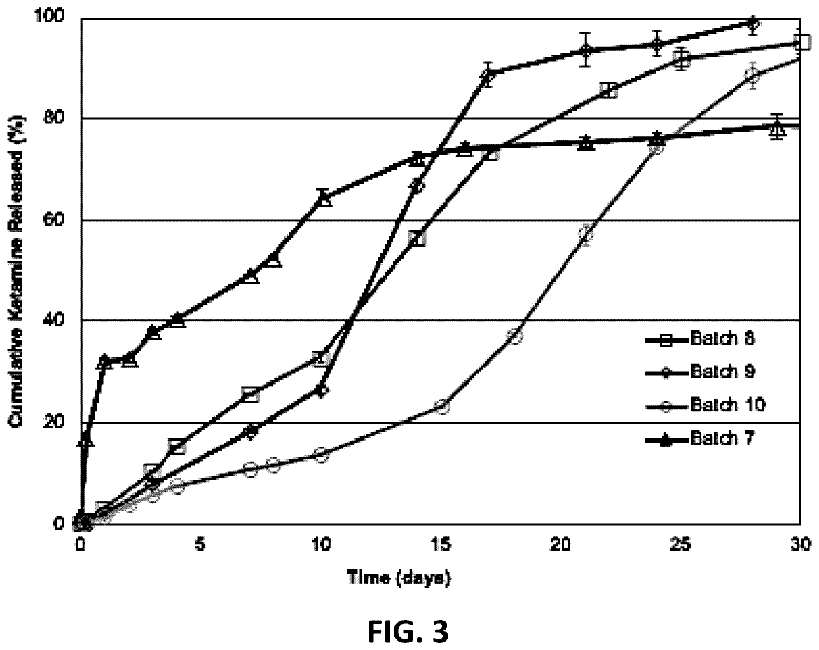

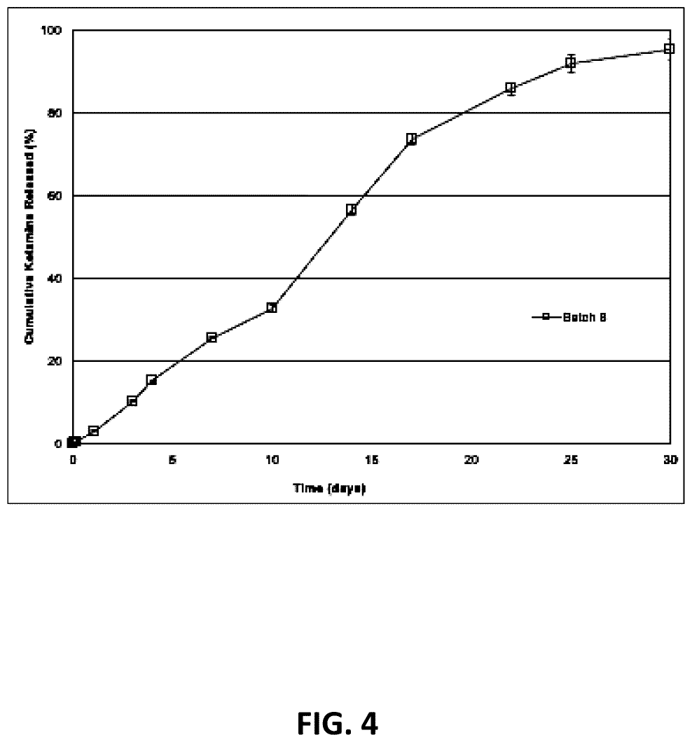

Batch No. 8 was tested in an in vitro assay mimicking physiological conditions. The cumulative percent release of ketamine over time is shown graphically in FIG. 3 (in comparison to other single and double emulsified batches, as a function of the inherent viscosity of the biodegradable polymer) and in FIG. 4.

### Example 3—Preparation and Evaluation of a Low Inherent Viscosity (0.66 dL/g) PLA-Based Double Emulsion Microsphere Formulation, Comprising PVA in the IA Component

Batch No. 9: The organic component was formed by dissolving 70.0 g of an ester end-capped PLA Ashland Viatel 07 E polymer (IV=0.66 dL/g) in 388 g of DCM and 46 g of ethanol (5:1 ratio, by volume), followed by the addition of esketamine (30.0 g) with mixing until completely dissolved. The organic component was homogenized with an IA component consisting of 11 mL of a 0.35% PVA solution in a T25 Ultra-turrax high-speed homogenizer operating at 21,500 rpm for 45 seconds to form the PE.

The PE was pumped into a Levitronix i100 operating at 1,600 rpm at a rate of 30 mL/minute, along with the CP comprising water and 0.35% PVA, which was pumped at a rate of 2 L/min, for a CP: PE ratio of 66:1.

The formed or forming microspheres exited the homogenizer and entered an SRV. Deionized water was added to the SRV at 2 L/min. Solvent removal was achieved by washing the microspheres with ambient water (i.e., 25° C.) and hot water (35-39° C.) and filtering them through a hollow fiber filter.

The bulk suspension was collected via filtration and lyophilized to obtain a free-flowing powder with a yield of about 72%. The drug load was 14.6 wt/wt % (49% drug encapsulation efficiency based on a target drug load of 30 wt/wt %). The average particle size was 38 μm (D₁₀), 75 μm (D₅₀), 123 μm (D₉₀).

Batch No. 9 was tested in an in vitro assay mimicking physiological conditions. The cumulative percent release of ketamine over time is shown graphically in FIG. 3 (in comparison to other single and double emulsified batches, as a function of the inherent viscosity of the biodegradable polymer).

### Example 4—Preparation and Evaluation of a Higher Inherent Viscosity (1.80 dL/g) PLA-Based Double Emulsion Microsphere Formulation

Batch No. 10: The organic component was formed by dissolving 7.0 g of an ester end-capped PLA Evonik LG 207S polymer (IV=1.80 dL/g) in 63 g of DCM and 4.6 g of ethanol (8:1 ratio, by volume), followed by the addition of esketamine (3.0 g) with mixing until completely dissolved. The organic component was homogenized with an IA component consisting of 1 mL of a 0.35% PVA solution in a T25 Ultra-turrax high-speed homogenizer operating at 21,500 rpm for 30 seconds to form the PE.

The PE was pumped into a Levitronix i100 operating at 1,600 rpm at a rate of 30 mL/minute, along with the CP comprising water and 0.35% PVA, which was pumped at a rate of 2 L/min, for a CP:DP ratio of 66:1.

The formed or forming microspheres exited the homogenizer and entered an SRV. Deionized water was added to the SRV at 2 L/min. Solvent removal was achieved by washing the microspheres with ambient water (i.e., 25° C.) and hot water (35-39° C.) and filtering them through a hollow fiber filter.

The bulk suspension was collected via filtration and lyophilized to obtain a free-flowing powder with a yield of about 56%. The drug load was 17.4 wt/wt % (58% drug encapsulation efficiency based on a target drug load of 30 wt/wt %). The average particle size was 27 μm (D₁₀), 67 μm (D₅₀), 136 μm (D₉₀).

Batch No. 10 was tested in an in vitro assay mimicking physiological conditions. The cumulative percent release of ketamine over time is shown graphically in FIG. 3 (in comparison to other single and double emulsified batches, as a function of the inherent viscosity of the biodegradable polymer).

### Example 5—Pharmacokinetics Study in Rats of Batch Nos. 7, 9, and 10

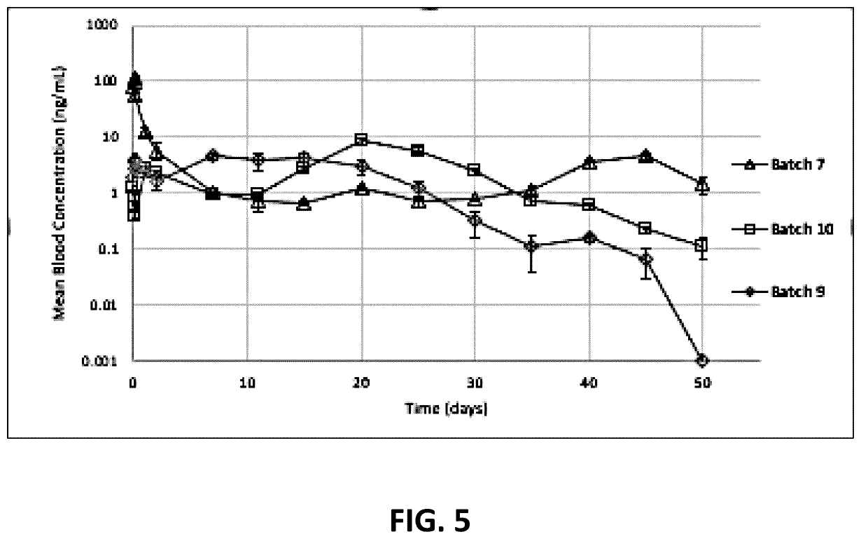

The pharmacokinetic profile of ketamine following a subcutaneously injected dose of time-released ketamine formulation in male Sprague-Dawly rats was studied. The rats received a 50 mg/kg dose of the indicated Batch No., having a ketamine concentration of 33.33 mg/mL and a volume of 1.5 ml/kg. Microsphere suspension concentrations (mg/mL) were as follows: (a) Batch No. 7:130.21; (b) Batch No. 9:228.31; and (c) Batch No. 10:191.57. Blood was collected at 0.5, 1, 2, 4, 24, 48, 168, 264, 360, 480, 600, 720, 840, 960, 1080, and 1200 hours. FIG. 5 is a graph showing the measured mean blood concentration (ng/ml) of ketamine as a function of time for Batches Nos. 7 (a single emulsion microsphere formulation), 9, and 10.

### Example 6—Low Inherent Viscosity (0.67 dL/g) PLA-Based Double Emulsion Microsphere Formulations, with a CP:PE Ratio of 100:1

Batch Nos. 11A and 11B: An organic component was formed by dissolving 14.0 g of an ester end-capped PLA Ashland DL Viatel 07 E polymer (IV =0.67 dL/g) in 77.58 g of DCM and 9.2 g of ethanol (5:1 ratio, by volume), followed by the addition of esketamine (6.0 g) with mixing until completely dissolved. The organic component was homogenized with an IA component consisting of 2.18 g of 0.35% PVA solution in a T25 Ultra-turrax high-speed homogenizer operating at 21,500 rpm for 30 seconds to form the PE having an organic:IA component ratio of about 49:1 (on a mass basis).

The primary emulsion was pumped into a Levitronix i100 operating at 1,600 rpm at a rate of 20 mL/minute, along with a CP comprising water and 0.35% PVA, which was pumped at a rate of 2 L/min, for a CP:PE ratio of 100:1.

The formed or forming microspheres exited the homogenizer and a portion (Batch No. 11A) of the suspension entered a first SRV, wherein the microspheres were subjected immediately to deionized water at 2 L/min. Solvent removal was achieved by washing the microspheres with ambient water (i.e., 25° C.) and hot water (35-39° C.) and filtering them through a hollow fiber filter. The bulk suspension was collected via filtration and lyophilized to yield 6.6 g of a free-flowing powder. The drug load was 23.0 wt/wt % (77% drug encapsulation efficiency based on a target drug load of 30 wt/wt %). The average particle size was 53 μm (D₁₀), 94 μm (D₅₀), 152 μm (D₉₀).

A second portion of the suspension (Batch No. 11B) entered a second SRV, wherein it was held for four hours. At the conclusion of the four hour hold, the microspheres were washed, filtered, and lyophilized as described with respect to Batch No. 11A to yield 6.7 g of a free-flowing powder. The drug load was 9.2 wt/wt % (31% drug encapsulation efficiency based on a target drug load of 30 wt/wt %). The mean particle size was 50 μm (D₁₀), 90 μm (D₅₀), 143 μm (D₉₀). The total yield of Batch Nos. 11A and 11B was 66.3%.

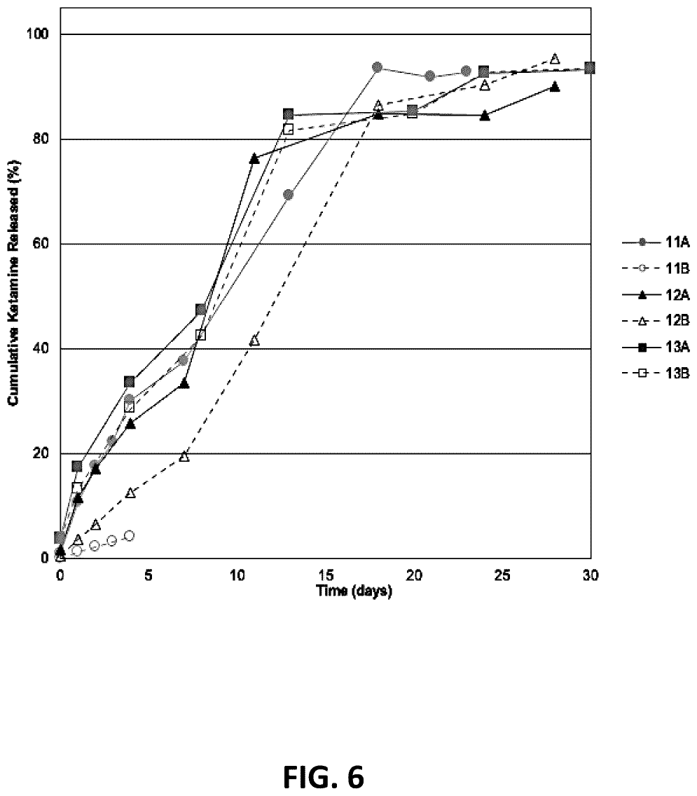

Batch Nos. 11A and 11B were tested in an in vitro assay mimicking physiological conditions. The cumulative percent release of ketamine over time is shown graphically in FIG. 6.

### Example 7—Low Inherent Viscosity (0.67 dL/g) PLA-Based Double Emulsion Microsphere Formulations, with a CP:PE Ratio of 80:1

Batch Nos. 12A and 12B: An organic component was formed by dissolving 10.5 g of an ester end-capped PLA Ashland DL 07 E polymer (IV=0.67 dL/g) in 58.19 g of DCM and 6.9 g of ethanol (5:1 ratio, by volume), followed by the addition of esketamine (4.5 g) with mixing until completely dissolved. The organic component was homogenized with an IA component consisting of 1.64 g of 0.35% PVA solution in a T25 Ultra-turrax high-speed homogenizer operating at 21,500 rpm for 30 seconds to form the PE having an organic:IA component ratio of about 49:1 (on a mass basis).

The PE was pumped into a Levitronix i100 operating at 1,600 rpm at a rate of 25 mL/minute, along with a CP comprising water and 0.35% PVA, which was pumped at a rate of 2 L/min, for a CP:PE ratio of 80:1.

The formed or forming microspheres exited the homogenizer and a portion (Batch No. 12A) of the suspension entered a first SRV, wherein the microspheres were subjected immediately to deionized water at 2 L/min. Solvent removal was achieved by washing the microspheres with ambient water (i.e., 25° C.) and hot water (35-39° C.) and filtering them through a hollow fiber filter.

The bulk suspension was collected via filtration and lyophilized to yield 0.89 g of a free-flowing powder. The drug load was 24.8 wt/wt % (83% drug encapsulation efficiency based on a target drug load of 30 wt/wt %). The mean particle size was 57 μm (D₁₀), 111 μm (D₅₀), 189 μm (D₉₀).

A second portion (Batch No. 12B) of the suspension entered a second SRV, wherein it was held for four hours. At the conclusion of the four hour hold, the microspheres were washed, filtered, and lyophilized as described with respect to Batch No. 12A to yield 7.2 g of a free-flowing powder. The drug load was 17.4 wt/wt % (58% drug encapsulation efficiency based on a target drug load of 30 wt/wt %). The mean particle size was 54 μm (D₁₀), 99 μm (D₅₀), 161 μm (D₉₀). The total yield of Batch Nos. 12A and 12B was 54%.

Batch Nos. 12A and 12B were tested in an in vitro assay mimicking physiological conditions. The cumulative percent release of ketamine over time is shown graphically in FIG. 6.

### Example 8—Low Inherent Viscosity (0.67 dL/g) PLA-Based Double Emulsion Microsphere Formulations, with a CP:PE Ratio of 80:1

Batch Nos. 13A and 13B: An organic component was formed by dissolving 10.5 g of an ester end-capped PLA Ashland DL 07 E polymer (IV=0.67 dL/g) in 58.19 g of DCM and 6.9 g of ethanol (5:1 ratio, by volume), followed by the addition of esketamine (4.5 g) with mixing until completely dissolved. The organic component was homogenized with an IA component consisting of 1.64 g of 0.35% PVA solution in a T25 Ultra-turrax high-speed homogenizer operating at 21,500 rpm for 30 seconds to form the PE having an organic:IA component ratio of about 49:1 (on a mass basis).

The PE was pumped into a Levitronix i100 operating at 1,600 rpm at a rate of 25 mL/minute, along with a CP comprising water and 0.35% PVA, which was pumped at a rate of 2 L/min, for a CP:DP ratio of 80:1.

The formed or forming microspheres exited the homogenizer and a portion (Batch No. 13A) of the suspension entered a first SRV, wherein the microspheres were subjected immediately to deionized water at 2 L/min. Solvent removal was achieved by washing the microspheres with ambient water (i.e., 25° C.) and hot water (35-39° C.) and filtering them through a hollow fiber filter.

The bulk suspension was collected via filtration and lyophilized to yield 2.99 g of a free-flowing powder. The drug load was 29.4 wt/wt % (98% drug encapsulation efficiency based on a target drug load of 30 wt/wt %). The mean particle size was 46 μm (D₁₀), 104 μm (D₅₀), 190 μm (D₉₀).

A second portion (Batch No. 13B) of the suspension entered a second SRV, wherein it was held for four hours. At the conclusion of the four hour hold, the microspheres were washed, filtered, and lyophilized as described with respect to Batch No. 13A to yield 7.09 g of a free-flowing powder. The drug load was 26.4 wt/wt % (88% drug encapsulation efficiency based on a target drug load of 30 wt/wt %). The mean particle size was 52 μm (D₁₀), 99 μm (D₅₀), 162 μm (D₉₀). The total yield of Batch Nos. 13A and 13B was 67%.

Batch Nos. 13A and 13B were tested in an in vitro assay mimicking physiological conditions. The cumulative percent release of ketamine over time is shown graphically in FIG. 6.

### Example 9—Low Inherent Viscosity (0.67 dL/g) PLA-Based Double Emulsion Microsphere Formulations, with CP (1.0% PVA) and a CP:PE Ratio of 80:1

Batch Nos. 14A and 14B: An organic component was formed by dissolving 12.45 g of an ester end-capped PLA Ashland DL 07 E polymer (IV=0.67 dL/g) in 70.74 g of DCM and 8.39 g of ethanol (5:1 ratio, by volume), followed by the addition of esketamine (2.55 g) with mixing until completely dissolved. The organic component was homogenized with an IA component consisting of 1.64 g of 0.35% PVA solution in a T25 Ultra-turrax high-speed homogenizer operating at 21,500 rpm for 30 seconds to form the PE having an organic: IA component ratio of about 57:1 (on a mass basis).

The PE was pumped into a Levitronix i100 operating at 1,600 rpm at a rate of 25 mL/minute, along with a CP comprising water and 1.0% PVA, which was pumped at a rate of 2 L/min, for a CP:PE ratio of 80:1.

The formed or forming microspheres exited the homogenizer and a portion (Batch No. 14A) of the suspension entered a first SRV, wherein the microspheres were subjected immediately to deionized water at 2 L/min. Solvent removal was achieved by washing the microspheres with ambient water (i.e., 25° C.) and hot water (35-39° C.) and filtering them through a hollow fiber filter.

The bulk suspension was collected via filtration and lyophilized to yield 2.99 g of a free-flowing powder. The drug load was 14.5 wt/wt % (85% drug encapsulation efficiency based on a target drug load of 17 wt/wt %). The mean particle size was 32 μm (D₁₀), 87 μm (D₅₀), 149 μm (D₉₀).

A second portion (Batch No. 14B) of the suspension entered a second SRV, wherein it was held for four hours. At the conclusion of the four hour hold, the microspheres were washed, filtered, and lyophilized as described with respect to Batch No. 14A to yield 6.95 g of a free-flowing powder. The drug load was 13.7 wt/wt % (81% drug encapsulation efficiency based on a target drug load of 17 wt/wt %). The mean particle size was 37 μm (D₁₀), 88 μm (D₅₀), 148 μm (D₉₀). The total yield of Batch Nos. 14A and 14B was 75%.

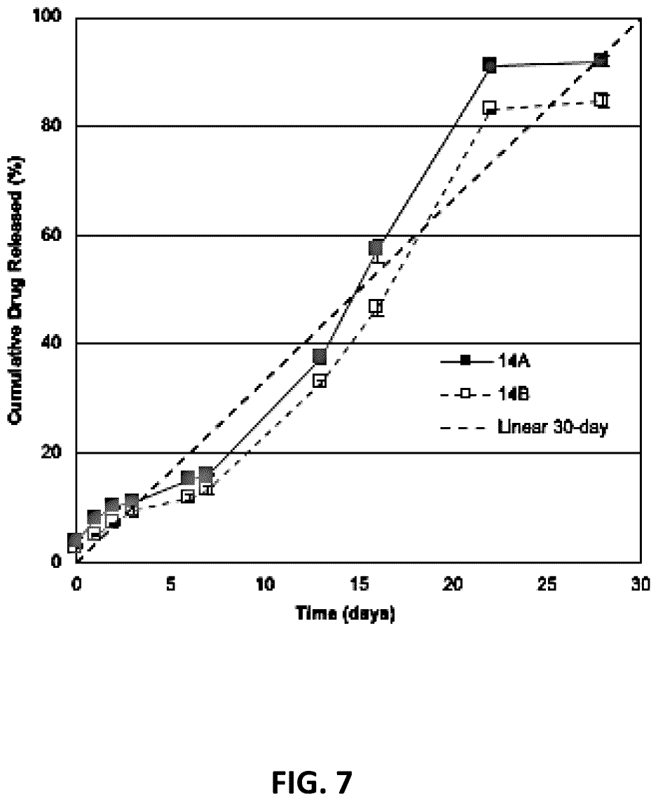

Batch Nos. 14A and 14B were tested in an in vitro assay mimicking physiological conditions. The cumulative percent release of ketamine over time is shown graphically in FIG. 7 compared to an ideal 30 day release profile.

### Example 10—Large Scale Preparation of PLA-Based Double Emulsion Microsphere Formulations

Batch Nos. 164 and 165: An organic component was formed by dissolving 972 g of an ester end-capped PLA Ashland DL 07 E polymer (Batch No.164: IV=0.67 dL/g; Batch No.165: IV −0.72 dL/g) in 5494 g of DCM and 652 g of ethanol (8.5:1 ratio, by weight), followed by the addition of esketamine (228 g) with mixing until completely dissolved. The IA component and the organic component were pumped into the sonicator at a flow rate of 0.52 mL/min and 24.5 mL/min, respectively, and the PE was pumped into a Levitronix i100 operating at 1,600 rpm at a rate of 25 mL/minute, along with a CP comprising water and 0.5% PVA, which was pumped at a rate of 2 L/min, for a CP:PE ratio of 80:1.

The formed or forming microspheres exited the homogenizer and the suspension entered an SRV, wherein it was held for four hours. At the conclusion of the four hour hold, the microspheres were washed, filtered, and lyophilized to obtain a free-flowing powder.

The microspheres of Batch No. 164 had a particle size of 90 μm (D₅₀) and a drug load of 14.4 wt % (76% encapsulation efficiency based on a target drug load of 19%).

The microspheres of Batch No. 165 had a particle size of 91 μm (D₅₀) and a drug load of 14.3 wt % (76% encapsulation efficiency based on a target drug load of 19%).

The process parameters and results for each batch are shown tabularly in Table 1:

| Lot | Batch | | 164 | 165 |
|---|---|---|---|---|
| | Number | | | |
| Polymer | Polymer | | Ashland DL07 E | |
| | Supplier/Name | | | |
| | Co-Monomer Ratio | | 100:0 | |
| | Polymer IV (dL/g) | | 0.67 | 0.72 |
| | Polymer Endcap | | Ester | |
| Formulations | Batch Size (g) | | 1200 | |
| | Mixing Speed | | 1600 | |
| | (RPM) | | | |
| | Target Drug Load | | 19 | |
| | (%) | | | |
| Analytical | Drug Load (%) | | 14.4 | 14.33 |
| | Encapsulation | | 76% | 76% |
| | Efficiency (%) | | | |
| | Particle | Dᵥ10 | 46 | 39 |
| | Size (μm) | Dᵥ50 | 90 | 91 |
| | | Dᵥ90 | 144 | 150 |

| | | |
|---|---|---|
| Sample MW (kDa) | 76 | 93 |
| Polymer MW (kDa) | 79 | 97 |

### Example 11—Pharmacokinetics Study in Dogs of Batch Nos. 164 and 165 (the “Dog PK Study Formulations”)

The pharmacokinetic profile of ketamine in dogs following a subcutaneously injected dose of the large scale batches from Example 10 was studied. The dogs received a 15 mg/kg dose of the indicated Batch No., having a ketamine concentration of 45.5 mg/mL and a volume of 0.33 mL/kg. Microsphere suspension concentrations (mg/mL) were as follows: (a) Batch No. 164: 315.7; and (b) Batch No. 165: 317.8. Blood was collected pre-dose, at 0.5, 1, 2, 4, 8, 12, 24, 48, 72, and 96 hours, and at 7, 10, 13, 17, 20, 23, 27, 30, 33, 37, 40, 45, and 50 days.

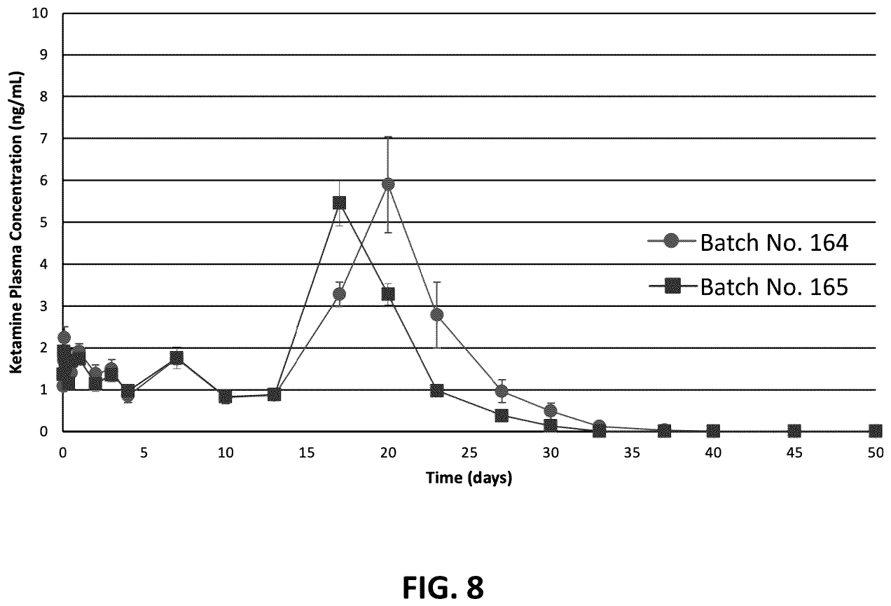

FIG. 8 is a graph showing the measured mean blood concentration (ng/ml) of ketamine as a function of time for the dog PK study formulations.

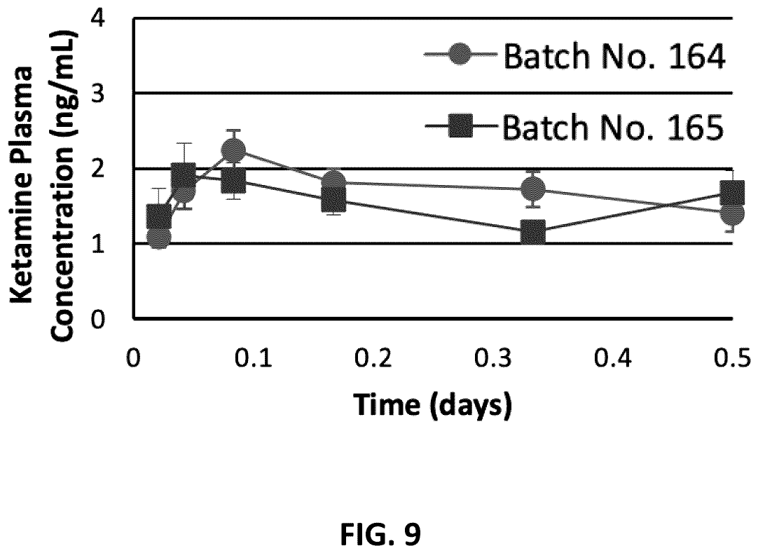

FIG. 9 is an inset graph depicting an expanded view of the first 0.5 day of the measurements shown in FIG. 8 and showing the burst release levels of ketamine. FIG. 9 shows that the polymer microspheres had negligible to no burst levels.

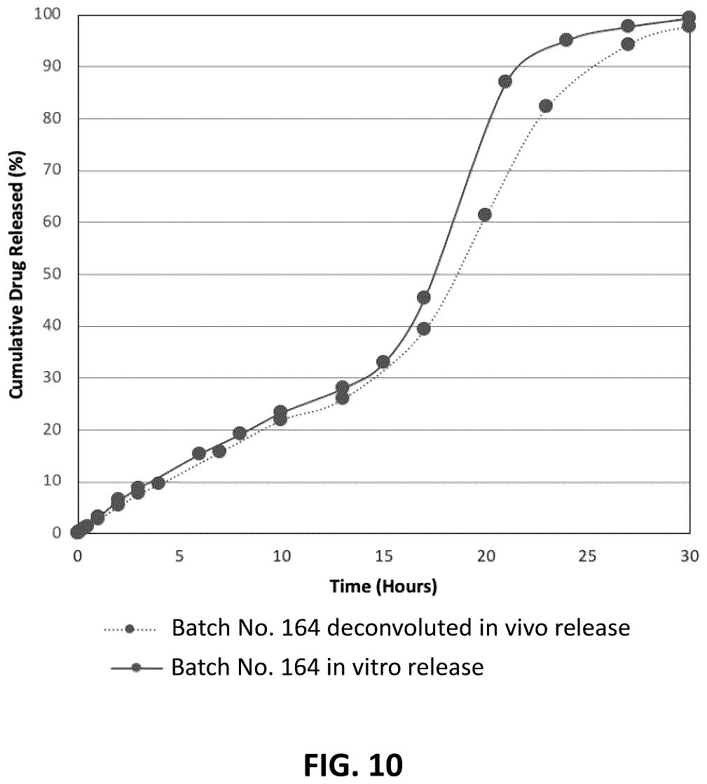

FIG. 10 is a graph showing the in vitro-in vivo correlation of polymer microspheres from Batch No. 164. The formulations exhibited good in vitro-in vivo correlation, and the curves showed the same general shape. All release profiles show full release within 30-33 days.

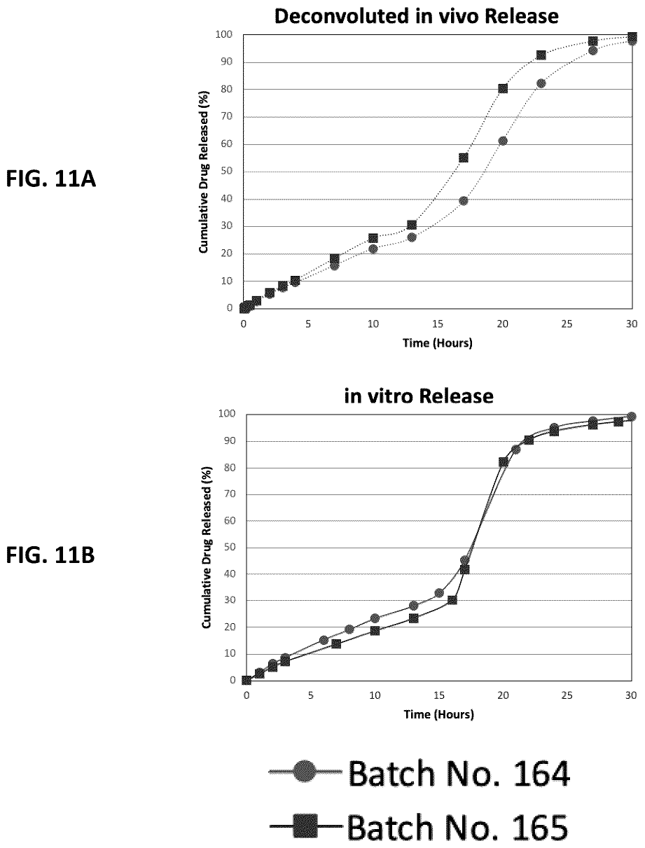

FIGS. 11A and 11B are graphs showing a comparison of deconvoluted in vivo release versus in vitro release of the dog PK study formulations. Again, the formulations exhibited good in vitro-in vivo correlation, and the curves showed the same general shape.

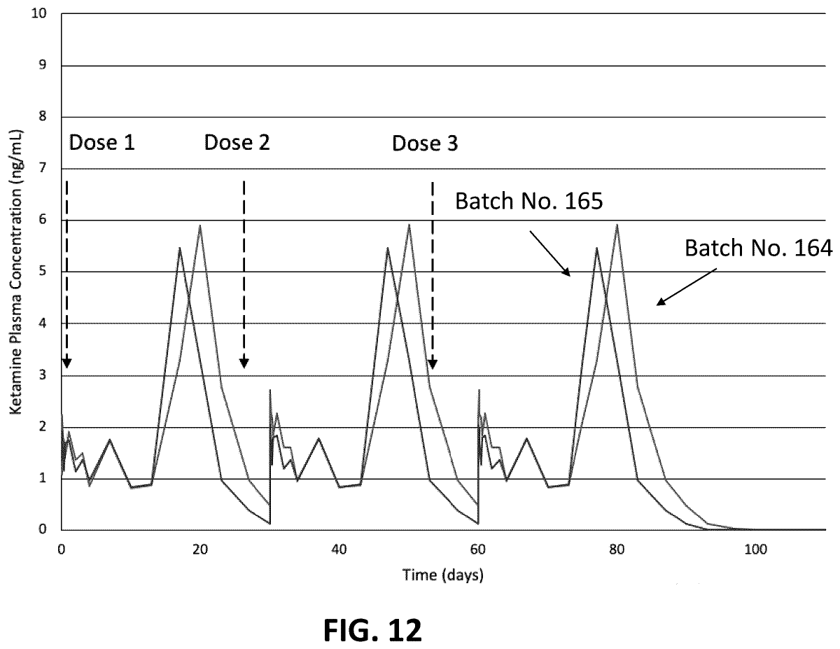

FIG. 12 is a graph showing simulated dosing of the dog PK study formulations over three months on a linear scale. The formulations do not exhibit accumulation of ketamine over time from successive doses.

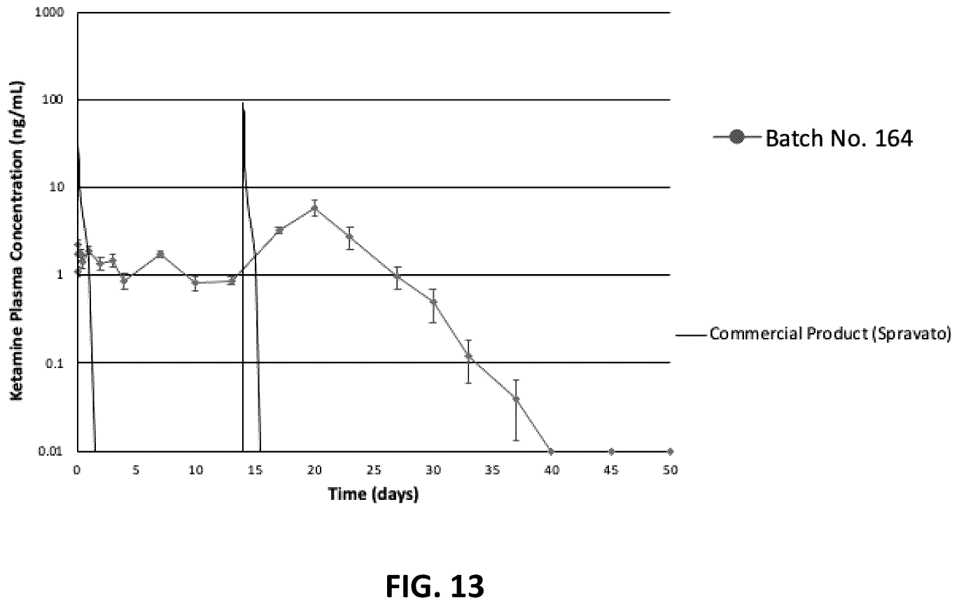

FIG. 13 is a graph showing a comparison of the in vivo release of Batch No. 164 in dogs versus the blood levels in humans resulting from nasal inhalation of the commercially available Spravato® nasal spray. See Center for Drug Evaluation and Research, Application Number 211243Orig1s000, Clinical Pharmacology and Biopharmaceutics Review(s), available at https://www.accessdata.fda.gov/drugsatfda_docs/nda/2019/211243Orig1s000ClinPharmR.pdf. Batch No. 164 shows no burst levels and consistent release over 30 days compared to Spravato® nasal spray, which has a high spike of drug and is then quickly metabolized and eliminated. Spravato® nasal spray requires close monitoring by a doctor for several hours after administration due to the high burst levels.

The aspects disclosed herein are not intended to be exhaustive or to be limiting. A skilled artisan would acknowledge that other aspects or modifications to instant aspects can be made without departing from the spirit or scope of the invention. The aspects of the present disclosure, as generally described herein and illustrated in the figures, can be arranged, substituted, combined, separated, and designed in a wide variety of different configurations, all of which are contemplated herein.

Unless otherwise specified, “a,” “an,” “the,” “one or more of,” and “at least one” are used interchangeably. The singular forms “a”, “an,” and “the” are inclusive of their plural forms. The recitations of numerical ranges by endpoints include all numbers subsumed within that range (e.g., 1 to 5 includes 1, 1.5, 2, 2.75, 3, 3.80, 4, 5, etc.). The terms “comprising” and “including” are intended to be equivalent and open-ended. The phrase “consisting essentially of”' means that the composition or method may include additional ingredients and/or steps, but only if the additional ingredients and/or steps do not materially alter the basic and novel characteristics of the claimed composition or method. The phrase “selected from the group consisting of” is meant to include mixtures of the listed group.

When reference is made to the term “each,” it is not meant to mean “each and every, without exception.” For example, if reference is made to microsphere formulation comprising polymer microspheres, and “each polymer microsphere” is said to have a particular ketamine content, if there are 10 polymer microspheres, and two or more of the polymer microspheres have the particular ketamine content, then that subset of two or more polymer microspheres is intended to meet the limitation.

The term “about” in conjunction with a number is intended to include #10% of the number. That is, the number is intended to be read as if it was followed by the phrase, “±10%”. This is true whether “about” is modifying a stand-alone number or modifying a number at either or both ends of a range of numbers. In other words, “about 10” means from 9 to 11. Likewise, “about 10 to about 20” contemplates 9 to 22 and 11 to 18. In the absence of the term “about,” the exact number is intended. In other words, “10” means 10.

What is claimed is:

1. A microsphere formulation, comprising:
polymer microspheres, each polymer microsphere comprising:
(1) esketamine; and
(2) a biodegradable poly(lactide) polymer having an inherent viscosity of about 0.6 dL/g to about 0.72 dL/g,
wherein each polymer microsphere has a ketamine drug load of about 12 wt/wt % to about 17 wt/wt %;
wherein the polymer microspheres have a particle size of about 80 μm (D₅₀) to about 110 μm (D₅₀); and
wherein the polymer microspheres are characterized in that each of the polymer microspheres comprises a plurality of internal macrovoids.

2. The microsphere formulation of claim 1, wherein the microsphere formulation is characterized in that about 75% to 100% of the ketamine is released over a period of between about 25 to about 35 days of injection into a human subject, but not more than about 10% of the ketamine has been released within 24 hours of injection into the human subject.

3. The microsphere formulation of claim 1, wherein the biodegradable poly(lactide) polymer has an inherent viscosity between about 0.67 dL/g and about 0.72 dL/g.

4. The microsphere formulation of claim 1, wherein each polymer microsphere has a ketamine drug load of about 13 wt/wt % to about 16 wt/wt %.

5. The microsphere formulation of claim 1, wherein the polymer microspheres have a particle size of about 90 (D₅₀).

6. A pharmaceutical composition comprising the microsphere formulation of claim 1.

7. A method for treating depression, the method comprising intramuscularly or subcutaneously injecting a patient in need thereof with a therapeutically effective amount of a microsphere formulation as claimed in claim 1.

8. A microsphere formulation, comprising:
polymer microspheres, each polymer microsphere comprising:
(i) ketamine; and
(ii) a biodegradable poly(lactide) polymer;
wherein each polymer microsphere has a ketamine drug load of about 10 wt/wt % to about 30 wt/wt %, and
wherein the polymer microspheres have a particle size greater than 60 um (D₅₀).

9. The microsphere formulation of claim 8, wherein the polymer microspheres have a particle size of about 80 μm (D₅₀) to about 110 μm (D₅₀).

10. The microsphere formulation of claim 8, wherein the ketamine comprises a free base form of esketamine.

11. The microsphere formulation of claim 8, wherein each polymer microsphere has a ketamine drug load of between about 12 wt/wt % to about 17 wt/wt %.

12. The microsphere formulation of claim 8, wherein the biodegradable poly(lactide) polymer has an inherent viscosity from about 0.6 dL/g to about 0.72 dL/g.

13. The microsphere formulation of claim 8, wherein the microsphere formulation is characterized in that about 75% to 100% of the ketamine is released over a period of between about 25 to about 35 days of injection into a human subject, but not more than about 10% of the ketamine has been released within 24 hours of injection into the human subject.

14. The microsphere formulation of claim 8, characterized in that each of the polymer microspheres comprises a plurality of internal emulsions comprising water and a surfactant.

15. The microsphere formulation of claim 8, characterized in that each of the polymer microspheres comprises a plurality of internal macrovoids.

16. A pharmaceutical composition comprising the microsphere formulation of claim 8.

17. A method for treating depression, the method comprising intramuscularly or subcutaneously injecting a patient in need thereof with a therapeutically effective amount of a microsphere formulation as claimed in claim 8.

18. A kit, comprising:
(A) a microsphere formulation, the microsphere formulation comprising:
polymer microspheres, each polymer microsphere comprising:
(1) esketamine; and
(2) an ester-terminated biodegradable poly(lactide) polymer having an inherent viscosity of about 0.67 dL/g,
wherein each polymer microsphere has a ketamine drug load of about 14 wt/wt % to about 15 wt/wt %;
wherein the polymer microspheres have a particle size of about 90 μm (D₅₀); and
wherein the polymer microspheres are characterized in that each of the polymer microspheres comprises a plurality of internal macrovoids; and
wherein the microsphere formulation is characterized in that about 75% to 100% of the ketamine is released over a period of between about 25 to about 35 days of injection into a human subject, but not more than about 10% of the ketamine has been released within 24 hours of injection into the human subject.

19. The kit of claim 18, further comprising a diluent.

20. The kit of claim 18, further comprising a syringe.
